(19) United States
(12) Patent Application Publication
(10) Pub. No.: US 2019/0269594 A1
(43) Pub. Date: Sep. 5, 2019

(54) COSMETIC COMPOSITION COMPRISING NATURAL ACTIVES

(71) Applicant: Conopco, Inc., d/b/a UNILEVER, Englewood Cliffs, NJ (US)

(72) Inventors: Vijay Ramchandra GADGIL, Bangalore (IN); Ramitha KALATHIL, Bangalore (IN); Rajkumar PERUMAL, Bangalore (IN); Ravi Kant SHUKLA, Bangalore (IN); Ashish Anant VAIDYA, Bangalore (IN)

(21) Appl. No.: 16/349,137

(22) PCT Filed: Oct. 30, 2017
(86) PCT No.: PCT/EP2017/077737

§ 371 Date: May 10, 2019

(30) Foreign Application Priority Data
Nov. 24, 2016 (EP) 16200546.6

(51) Int. Cl. A61K 8/49; A61Q 17/04

(52) U.S. Cl. CPC A61K 8/4973; A61K 8/498; A61K 2800/49; A61K 2800/30; A61Q 17/04

(57) ABSTRACT

Disclosed is a cosmetic composition comprising: (i) 0.1 to 10 % by weight pongamol; (ii) 0.1 to 10 % by weight karanjin; and, (iii) 0.1 to 40% by weight of a solvent whose Total Hildebrand Parameter (δt) value is 18 to 26 MPa^{0.5} at 25° C., wherein said composition comprises not more than 2% by weight pongamia oil. The composition provides abrasion resistant photoprotection to skin.

### FIELD OF THE INVENTION

The invention relates to a cosmetic composition comprising natural actives, especially to compositions which resist wear-off due to abrasion.

### BACKGROUND OF THE INVENTION

Cosmetic compositions such as creams and lotions are applied to get maximum protection or any other desired effect. Generally, such compositions are applied by first dispensing a desired quantity on the palm of one hand followed by gentle rubbing of the composition on the desired part of the body using both hands. Compositions like hand and body lotions are applied in liberal amounts whereas just a little amount may be sufficient for some other types.

Any user would expect the composition to remain in contact with skin, such as arms or the face, for as long as possible. However, there are certain natural and some man made factors that may not let this happen easily.

Sweat, wind and moisture are some important natural factors. On the other hand, contact with clothes, which in many cases, is inevitable, is one of the man-made factors. Such contact leads to abrasion, which may not be sufficient to cause discomfort to a wearer or for that matter, may not even be noticeable, but it may be sufficient to wear-off or rub-off the film of the cosmetic composition which is formed on the skin.

Solar radiation contains about 5% ultraviolet (UV) radiation. The radiation is classified into three sub-regions; from 320 to 400 nm (UV-A), 290 to 320 nm (UV-B) and from 200 to 290 nm (UV-C). Scientific studies have indicated that exposure to UV-A and UV-B radiation for short period causes reddening of the skin and localized irritation, whereas continued and prolonged exposure could lead to sunburn, melanoma wrinkles. Therefore, it is desirable to protect the skin from harmful effects of solar radiation. Various cosmetic preparations have been reported for protecting the skin from harmful effects of ultraviolet radiation. Numerous organic sunscreen agents capable of absorbing UV-A and or UV-B radiation are known in the field of cosmetics.

Pongamol and karanjin are two naturally occurring compounds isolated from Pongamia species and they, respectively, are UV-A and UV-B sunscreens.

IN 1307/DEL/2009 A (BEHL HARI MOHAN) discloses the use of 4% extract of Pongamia pinnata with many other herbs in a cosmetic.

IN 2600/DEL/2014 A [Tribal Cooperative Marketing Development Federation of India Ltd, TRIFED] discloses personal care compositions such as liquid soap, cold cream, sunscreen and lather shaving cream comprising 1 to 25% seed oil of Pongamia species in a physiologically acceptable excipient. The compositions exemplified in this publication contain from 7 to 13% by weight pongamia oil.

IN 3104/MUM/2014 A [Kancor Ingredients Ltd] discloses pongamol in high yield of at least 2% isolated from karanja oil and karanja oil extract having high pongamol content of at least 5%. Karanja oil is extracted with ethanol in the w/v ratio 1:1 at 50 to 60° C. Ethanol is removed and the extract is treated with concentrated organic acid in the weight ratio 5 to 1000 and at 50 to 120° C. to obtain karanja oil extract having high pongamol content.

FR2972346 A1 (L'OREAL) discloses use of 2-methyl succinic acid di-ester derivative as solvent in cosmetic compositions comprising 5% by weight pongamol.

WO 2015145464 A2 (Kancor Ingredients Ltd) discloses a sunscreen composition containing 1 to 5% by weight dibenzoylmethane derivative, 1 to 10% by weight octyl-methoxy cinnamate and 0.9 to 6% by weight pongamol. It is disclosed that pongamol, by virtue of being a natural sunscreen, increases the UV absorption efficiency and maintains it due to photostability imparted it.

WO 2014114888 A2 discloses uses of Pongamol (1%) and Pongamia seed oil (20%) in a cosmetic composition along with acrylate-styrene copolymer.

WO14016349 A1 [Biosynthis] discloses a photoprotective composition containing karanja oil and at least one polyester resulting from the reaction between a C20-C44 carboxylic acid dimer and a polyol. The compositions disclosed in this application confer SPF of at least 20 and effective protection against UVA rays and contain 6 to 65% by weight karanja oil (which is another name for pongamia oil), depending on the nature of the cosmetic composition.

WO2016016540 A2 (Innovi) discloses a synergistic combination of at least two agents promoting the photoprotective effectiveness of a sunsceen. The first agent is a furanoflavone e.g., pongamol, which protects against UVA and the other is phenolic acid or derivative thereof which protect against UVB.

U.S. Pat. No. 5,152,983 A (Unilever, 1992) discloses several cosmetic compositions containing 0.5 to 2.0% by weight pongamol.

Thus, it is clear that all the formulations disclosed in the art contain pongamol oil as one of the ingredients. The primary reason for doing so is the ease of availability of the oil coupled with the complexity and lack of commercial viability of separating the oil into its components to get the active ingredients.

Compositions containing pongamol oil are difficult to process or manufacture on a commercial scale. Further, the oil may present the problem of instability or incompatibility with other ingredients generally present in cosmetic compositions. Addition of stabilisers or solubilising solvents is not a practical solution.

### SUMMARY OF THE INVENTION

It has been determined that such compositions present at least some technical problems.

The compositions are prone to abrade or rub-off on application therefore they are less likely to remain in contact with skin for at least the minimum amount of time. Such a problem is likely to affect the credibility of certain compositions like sun protection compositions, which are intended to remain in contact with skin for long periods.

Surprisingly, a solution to this problem lies in a solvent having certain specific properties.

Disclosed in accordance with a first aspect of the invention is a cosmetic composition comprising:
(i) 0.1 to 10% by weight pongamol;
(ii) 0.1 to 10% by weight karanjin; and,
(iii) 0.1 to 40 wt % of solvent whose Total Hildebrand Parameter (δt) is 18 to 26 MPa^{0.5} (at 25° C.),
   wherein said composition comprises not more than 2% by weight pongamia oil.

These and other aspects, features and advantages will become apparent to those of ordinary skill in the art from a reading of the following detailed description and the appended claims. For the avoidance of doubt, any feature of one aspect of the present invention may be utilised in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

All references to the term/expression wt % or % by weight, shall mean percentage by weight of the composition, except where indicated otherwise.

### DETAILED DESCRIPTION OF THE INVENTION

### Pongamol and Karanjin

Pongamia pinnata also called as Derris indica, is a monotypic genus which grows abundantly in India and neighboring countries. There are some more species of Pongamia but the glabra is well known. Phytochemical investigation of Pongamia pinnata reveals the presence of flavonoids such as furanoflavones, furanoflavonols, chromenoflavones, furanochalcones and pyranochalcones. The seed oil of Pongamia species contains acids like oleic acid, stearic acid and palmitic acid and whereas active ingredients like pongamol and karanjin, which are natural sunscreens, which are present in minor amounts.

Pongamol is also known as 1,3-propanedione, 1-(4-methoxy-5-benzofuranyl)-3 phenyl. Its structure can be found e.g., in U.S. Pat. No. 5,152,983 B (Chesebrough-Pond's USA Co., 1992), which is as follows:

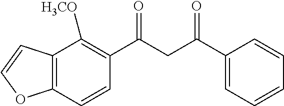 [SMILES: COc1c(C(=O)CC(=O)c2ccccc2)ccc2occc12]

Karanjin (3-methoxy-2-phenylfuro[2,3-h]chromen-4-one) is a furanoflavonol, a type of flavonoid, and is another active ingredient present in pongamia oil. Its structure is as follows:

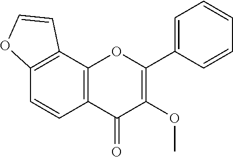 [SMILES: COc1c(-c2ccccc2)oc2c(ccc3occc32)c1=O]

Parmar et. al. have, in an article published by the Indian Agricultural Research Institute, reported that karanjin content in pongamia oil is about 1.25% by weight whereas pongamol is about 0.85% by weight.

Pavithra et. al. have, in J Am Oil Chem Soc (2012) 89:2237 to 2244 reported karanjin content varying from 3 to about 5% by weight of the pongamia oil.

The website http://www.biosynthis.com/#!karansun/c1n3e discloses KARANSUN™, a natural vegetable oil issued from Indian biodiversity. It is disclosed that pongamia seed oil contains two molecules, pongamol and karanjin which are natural UV-A and UV-B absorbers. Pongamol's structure similar to Avobenzone (Parsol 1789).

There are obvious limits on the amount of pongamol and karanjin that a formulation scientist can incorporate in conventional manner through pongamol oil.

As discussed earlier, the problem of abrasion resistance manifests itself if the active agents are used as such.

A solution lies in solvents having specific property.

The compositions in accordance with this invention comprise 0.1 to 10% by weight pongamol. Preferably the compositions comprise 0.5 to 5% by weight pongamol. More preferably the compositions comprise 0.5 to 2% by weight pongamol. The amount of pongamol may vary depending on the extent of UV-A protection desired, the nature of the composition and the presence or absence of other natural or synthetic UV sunscreens.

The compositions in accordance with this invention comprise 0.1 to 10% by weight karanjin. Preferably the compositions comprise 0.5 to 5% by weight karanjin. More preferably the compositions comprise 0.5 to 2.5% by weight karanjin. The amount of karanjin may vary depending on the extent of UV-B protection desired, the nature of the composition and the presence or absence of other natural or synthetic UV sunscreens.

In order to derive optimal benefits from pongamol and karanjin, it is preferred that the w/w ratio of pongamol to karanjin in the compositions according to the invention is 1:0.5 to 1:5. More preferably, this ratio is 1:1.5 to 1:3. The ratio will depend on the nature and type of the cosmetic composition and the presence or absence of other natural or synthetic sunscreens.

Composition in accordance with the invention comprise not more than 2% by weight pongamia oil, preferably not more than 1% and more preferably not more than 0.5% by weight pongamia oil. Most preferably the compositions in accordance with the invention are free of pongamia oil. The term “free of pongamia oil” means that the composition contains no more than traces of pongamia oil which may be present as impurity. The term pongamia oil may be used interchangeably with karanja oil. Both mean the same. Likewise the term pongamia oil is sometimes also referred to as pongamol oil.

### Solvent

Compositions in accordance with the invention comprise 0.1 to 40% by weight solvent whose Total Hildebrand Parameter (δt) is 18 to 26 MPa^{0.5} (at 25° C.). The term solvent as used herein excludes water. It is preferred that compositions in accordance with the invention comprise a solvent whose Total Hildebrand Parameter (δt) is 20 to 25 MPa^{0.5} (at 25° C.).

Preferably, the solvent is at least one of phenoxyethanol or phenethyl benzoate. Other solvents like isopropyl myristate, volatile silicones, non-volatile silicones and mineral oil are not suitable because their corresponding value is less than 18 MPa^{0.5}.

The total Hildebrand parameter (symbol: δt) is a result of the following intermolecular forces, so called Van der Waals forces: dispersion (δd), dipole-dipole (δp) and hydrogen bonding (δh). The total Hildebrand parameter can be calculated from these components as follows: δt=[δd²+δp²+δh²]^{0.5}

The table below shows the Total Hildebrand Parameter (δt) of two solvents within the scope of the present invention and one outside the scope. The values were calculated using the software referred earlier.

| | | Dipole- | Hydrogen | Total Hildebrand |
|---|---|---|---|---|
| | Dispersion | Dipole | bonding | Parameter |
| Sample | δd | δp | δh | δt/MPa^{0.5} |
| Phenoxyethanol | 18.4 | 7.6 | 13.2 | 23.9 |
| Phenethyl benzoate | 19.5 | 5.8 | 4.5 | 20.8 |
| Isopropyl myristate | 16.2 | 2.3 | 3.8 | 16.8 |

### Other Ingredients and Features of the Composition

“Personal Care Composition” as used herein, is meant to include a composition for topical application to sun-exposed areas of the skin and/or hair of humans. Such a composition may be classified as leave-on or rinse off, and includes any product applied to a human body for improving appearance, cleansing, odour control or general aesthetics. The composition of the present invention can be in the form of a liquid, lotion, cream, foam, scrub, gel, or toner. Non-limiting examples of such compositions include leave-on skin lotions, creams, antiperspirants, deodorants, foundations, mascara and sunscreen lotions. The compositions of the present invention are preferably a leave-on composition, because such compositions pose challenges in terms of enhancement of photoprotection.

“Skin” as used herein is meant to include skin on the face and body (e.g., neck, chest, back, arms, underarms, hands, legs and scalp) and especially to the sun exposed parts thereof. Compositions of the invention is also of relevance to applications on any other keratinous substrates of the human body other than skin e.g. hair where products may be formulated with specific aim of improving photoprotection.

### Form of the Composition

It is preferred that compositions of the present invention are non-solid. They further preferably are non-solid leave on compositions. For the purpose of distinction, a solid composition implies a composition like a bar or cake of soap. The non-solid compositions include creams gels and lotions. Leave-on compositions are applied to the skin to remain in contact with skin for prolonged period. The leave-on compositions are distinguishable from rinse-off compositions which are applied and subsequently removed by washing, rinsing or wiping. Surfactants typically used for rinse-off compositions have physico-chemical properties giving them the ability to generate foam/lather in-use with ease of rinse; they can consist of mixtures of anionic, cationic, amphoteric, and non-ionic. Surfactants used in leave-on compositions on the other hand are not required to have such properties.

The compositions in accordance with the invention, preferably are emulsions of the oil-in-water type. Alternatively, they are emulsions of the water-in-oil type. In such cases, they preferably are the water-in-silicone type of emulsions. Creams and lotions are the most preferred formats. Further, alternatively the compositions are multiple emulsions of the type, e.g., oil-in-water-in-oil.

Non-solid (liquid) compositions of the invention are non-solid, meaning that that the composition has a measurable viscosity (measurable for instance with a Brookfield Viscometer DV-I (20 RPM, RV6, 30 Seconds, 20° C.) in the range of from 1 Pa·s. to 500 Pas, preferably from 2 Pa·s. to 100 Pas, more preferably from 3 Pas to 50 Pa·s. The compositions have critical shear stress (apparent yield stress) of less than 100 Pa, preferably less than 20 Pa at 25° C. The apparent yield stress is preferably at least 5 Pa at 25° C.

A lotion according to the present invention preferably has a viscosity of 1500 to 6000 cP as measured by Brookfield® Viscosity meter using LV #4, 30 rpm, 30° C. The definition of lotion has been given by Brummer in the book “Rheology Essentials of Cosmetic and Food Emulsions, Springer-Verlag Berline Heidelberg, 81-83 (2006)”. Therein lotions are compositions which flow out from a container at 25° C. when turned upside down.

Compositions of the present invention may further comprise a cosmetically acceptable vehicle to act as diluents, dispersants and/or carriers for the active ingredients used in the composition, so as to facilitate their distribution when the composition is applied to the skin. The cosmetically acceptable vehicle suitable for use in the present invention may be aqueous, anhydrous or an emulsion. Whenever present, water makes up for 20 to 80% by weight, more preferably 30 to 70% by weight of the compositions. Alternatively, but less preferably, the compositions in accordance with this invention are non-aqueous.

Besides water or in addition to water, organic solvents may also serve as carriers within compositions of the present invention. Such solvents are to be distinguished from the solvents which have a particular Total Hildebrand Parameter as defined in the statement of the invention.

One or more emollients may also be used as cosmetically acceptable carriers. Emollients are generally in the form of silicone oils and synthetic esters. Silicone oils may be volatile and non-volatile. Volatile silicone oils are preferably chosen from cyclic or linear polydimethylsiloxanes containing from 3 to 9, preferably from 4 to 5, silicon atoms. Non-volatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes.

Ester emollients that may be used are:
(a) Alkenyl or alkyl esters of fatty acids having 10 to 20 carbon atoms.
   Examples thereof include isoarachidyl neopentanoate, isononyl isonanonoate, oleyl myristate, oleyl stearate, and oleyl oleate.
(b) Ether-esters such as fatty acid esters of ethoxylated fatty alcohols.
(c) Polyhydric alcohol esters. Ethylene glycol mono- and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(d) Wax esters such as beeswax, spermaceti, myristyl myristate, stearyl stearate and arachidyl behenate.
(e) Sterols esters, of which cholesterol fatty acid esters are examples.

Emollients may be present in the composition anywhere from 0.1 to 50% by weight, preferably from 1 to 20% by weight of the composition.

### Oil-Soluble UV-B Sunscreen

It is preferred that the compositions of the invention comprise 1 to 10% by weight oil-soluble UV-B sunscreen. These are sunscreens other than karanjin. By oil-soluble is meant those UV-B sunscreens which are not soluble in water. It is preferred that compositions in accordance with the invention comprise 2 to 8% by weight of such sunscreens.

Preferably the oil-soluble UV-B sunscreen is at least one of octyl salicylate, 3,3,5-trimethylcyclohexyl 2-hydroxybenzoate, ethylhexyl salicylate, 2-ethylhexyl 2-cyano-3,3-diphenyl-2-propenoate, 2-ethylhexyl-4-methoxycinnamate or octylmethoxycinnamate, more preferably 2-ethylhexyl-4-methoxycinnamate. Some of the well known ingredients are Octisalate®, Homosalate®, Neo Heliopan®, Neo Heliopan® AV, Neo Heliopan® OS, Octocrylene® and Parsol® MCX. The oil-soluble UV-B sunscreen has Amax from 280 to 320 nm.

### Water-Soluble UV-B Sunscreen

In addition to the oil-soluble ones, or instead of the oil-soluble ones, the compositions of the invention may comprise 1 to 10% by weight water-soluble UV-B sunscreen. By water-soluble is meant those UV-B sunscreens which are not soluble in oil. It is preferred that compositions in accordance with the invention comprise 2 to 8%, more preferably 3 to 5% by weight of such sunscreens. Preferably the water-soluble UV-B sunscreen is at least one of 2-phenylbenzimidazole-5-sulfonic acid Na salt, benzophenone-4 or terephthalylidene dicamphor sulfonic acid. The water-soluble sunscreen has λmax from 280 and 400 nm.

A particularly preferred combination is where the oil-soluble UV-B sunscreen is 2-ethylhexyl-4-methoxycinnamate and the water-soluble UV-B sunscreen is 2-phenylbenzimidazole-5-sulfonic acid Na salt.

### Organic UV-A Sunscreen

Compositions in accordance with the invention comprise not more than 5% by weight organic UV-A sunscreen. These are sunscreens other than pongamol. Such a sunscreen is at least one of t-butylmethoxy dibenzoylmethane, 2-methyldibenzoylmethane, 4-methyl-dibenzoyl-ethane, 4-isopropyldibenzoyl-methane, 4-tert-butyldibenzoylmethane, 2,4-dimethyldibenzoylmethane, 2,5-dimethyldibenzoylmethane, 4,4′-diisopropyl-dibenzoylmethane, 2-methyl-5-isopropyl-4′-methoxydi benzoylmethane, 2-methyl-5-tert-butyl-4′-methoxy-dibenzoyl methane, 2,4-dimethyl-4′-methoxy dibenzoylmethane or 2,6-dimethyl-4-tert-butyl-4′-methoxy-dibenzoylmethane, bisdisulizole disodium, diethylamino hydroxybenzoyl hexyl benzoate, terephthalylidene dicamphor sulfonic acid or methyl anthranilate. Other UV-A sunscreens include but are not limited to Bisdisulizole disodium (Neo Heliopan® AP), Diethylamino hydroxybenzoyl hexyl benzoate (Uvinul® A Plus), Ecamsule® (Mexoryl® SX), Methyl anthranilate. The UV-A sunscreen has Amax from 330 to 380 nm.

When amount of such sunscreens is beyond the upper limit, the compositions become unstable presumably due to phase separation.

However, it is particularly preferred that compositions according to the invention comprises not more than 2% by weight synthetic organic suncreens and more preferably do not contain more than 1% by weight synthetic sunscreens.

### Crosslinked Silicone Elastomer

Compositions of the present invention preferably further comprise 1.5 to 10% by weight, more preferably 2 to 8% by weight crosslinked silicone (organopolysiloxane) elastomer. No specific restriction exists as to the type of curable organopolysiloxane composition that can serve as starting material for the crosslinked silicone elastomer. The elastomer serves as or forms the continuous phase of the water-in-silicone chassis. These weight values exclude any solvent such as cyclomethicone found in commercial “elastomer” silicones such as the Dow Corning products 9040 and 9045. For instance, the amount of crosslinked silicone elastomer in 9040 and 9045 is between 12 and 13% by weight, therefore the formulation will include an amount of DC 9045 equivalent to the amount of elastomer intended.

The crosslinked siloxane elastomer is either emulsifying or non-emulsifying type or is a combination thereof. The term “non-emulsifying,” as used herein, defines crosslinked organopolysiloxane elastomer from which polyoxyalkylene units are absent. The term “emulsifying,” as used herein, means crosslinked organopolysiloxane elastomer having at least one polyoxyalkylene (e.g., polyoxyethylene or polyoxypropylene) unit.

Particularly useful emulsifying elastomers are polyoxyalkylene-modified elastomers formed from divinyl compounds, particularly siloxane polymers with at least two free vinyl groups, reacting with Si—H linkages on a polysiloxane backbone. Preferably, the elastomers are dimethyl polysiloxanes crosslinked by Si—H sites on a molecularly spherical MQ resin.

Preferred silicone elastomers are organopolysiloxane compositions available under the INCI names of dimethicone/vinyl dimethicone crosspolymer, dimethicone crosspolymer and Polysilicone-11. Ordinarily these materials are provided as a 1-30% crosslinked silicone elastomer dissolved or suspended in a dimethicone fluid (usually cyclomethicone). For purposes of definition, “crosslinked silicone elastomer” refers to the elastomer alone rather than the total commercial compositions which also include a solvent (e.g., dimethicone) carrier.

Dimethicone/vinyl dimethicone crosspolymers and dimethicone crosspolymers are available from a variety of suppliers including Dow Corning (9040, 9041, 9045, 9506 and 9509), General Electric (SFE 839), Shin Etsu (KSG-15,16,18 [dimethicone/phenyl vinyl dimethicone crosspolymer]), and Grant Industries (Gransil® line of materials), and lauryl dimethiconetvinyl dimethicone crosspolymers supplied by Shin Etsu (e.g, KSG-31, KSG-32, KSG-41, KSG42, KSG-43, and KSG44).

Other suitable commercially available silicone elastomer powders include vinyl dimethicone/methicone silesquioxane crosspolymers from Shin-Etsu sold as KSP-100, KSP-101, KSP-102, KSP-103, KSP-104, KSP-105, and hybrid silicone powders that contain a fluoroalkyl group or a phenyl group sold by Shin-Etsu as respectively KSP-200 and KSP-300.

Most preferred as the silicone elastomer is DC 9045, which has a D5 cyclomethicone swelled elastomer particle size (based on volume and calculated as spherical particles) which averages about 38 micron, and may range from about 25 to about 55 micron.

Silicone elastomers differ from linear polymers due to cross-linking. Many silicone elastomers are made from linear silicone polymers that contain reactive sites along the polymer chain. Elastomers have different physical and chemical properties from linear polymers, and the properties of elastomers depend very much on the number of cross-links. An elastomer with lesser cross-linking will be very soft and will swell significantly in the presence of a compatible solvent. As the cross-linking increases, the hardness of the elastomer increases, and the elastomer will swell to a lesser extent in the presence of solvent. A highly suitable silicone elastomer for use in the composition of the invention is DC 9045, a dimethicone crosspolymer commercially available from Dow Corning. DC 9045 is chemically a blend of cyclopentasiloxane swelling agent and dimethicone crosspolymer (12 to 13%).

The swelling agent contained in the elastomer formulation is most preferably a silicone fluid or a functional silicone fluid. The swelling agent is preferably used in an amount, which is in a weight ratio of 1:10 to 10:1, more preferably 1:1 to 5:1 with respect to the reaction mixture where the silicone elastomer is prepared. Swelling agent is most preferably low molecular weight silicone oil which includes (i) low molecular weight linear and cyclic volatile methyl siloxanes, (ii) low molecular weight linear and cyclic volatile and non-volatile alkyl and aryl siloxanes, and (iii) low molecular weight linear and cyclic functional siloxanes. Most preferred, however, are low molecular weight linear and cyclic volatile methyl siloxanes (VMS). By “low molecular weight” in this paragraph is meant a compound having a molecular weight from 1000 to 9000 Daltons.

Other useful silicone elastomer blends which may be used in the present invention are commercially available as (DC 9027 (a blend of an ultra high viscosity dimethiconol and silicone elastomer in cyclopentasiloxane) available from Dow Corning, DC 9546 (a blend of high molecular weight silicone elastomer, cyclopentasiloxane and a high molecular weight linear silicone polymer) available from Dow Corning, EL8050 (a blend of high molecular weight polyglycol-modified silicone elastomer in isododecane) available from Dow Corning and EL8051 (a blend of high molecular weight polyglycol-modified silicone elastomer in isodecyl neopentanoate) available from Dow Corning.

### Emulsifier

Compositions in accordance with this invention may preferably comprise 0.5 to 5% by weight, more preferably 0.8 to 2% by weight emulsifier which is non-alkylated polyalkyleneglycol-modified dimethicone.

It is preferred that the emulsifier is at least one of polydimethylsiloxane polyether copolymer with pendant polyethylene oxide side chains, polydimethylsiloxane polyether copolymer with pendant polypropylene oxide side chains, polydimethylsiloxane polyether copolymer with pendant mixed polyethylene oxide and polypropylene oxide side chains, polydimethylsiloxane polyether copolymer with pendant mixed poly(ethylene)(propylene)oxide side chains. Most preferred is PEG-10 Dimethicone available from Shin Etsu under the trade name KF 6017® and its equivalents offered by Shin Etsu and other companies.

On the other hand, the emulsifiers not suitable for the present invention are and include Cetyl Diglyceryl Tris (Trimethyl-siloxy) silylethyl Dimethicone, Lauryl PEG-10 Tris (Trimethyl-siloxy) silylethyl Dimethicone, Lauryl PEG/PPG-18/18 Methicone, Cetyl PEG/PPG-10/1 Dimethicone and their equivalents. This is because such emulsifiers are alkylated.

### Humectants

Humectants of the polyhydric alcohol-type can be employed as cosmetically acceptable carriers. Preferably the humectant is at least one of propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, glycerol, ethoxylated glycerol, propoxylated glycerol. It is preferred that the amount of humectant is 6 to 12% by weight of the composition. Glycerol is the most preferred humectant.

### Hydrophobic Zinc Oxide

Compositions in accordance with this invention may additionally comprise 4 to 20% by weight hydrophobic zinc oxide. It may include an aqueous or non-aqueous carrier with zinc oxide particles dispersed therein. The zinc oxide particles have primary particle size of 100 nm or greater with substantially no or few zinc oxide particles having a particle size less than 100 nm. The zinc oxide particles could have a primary particle size of about 120 nm or greater, or about 150 nm or greater, or about 200 nm or greater.

### Other Ingredients

In addition to the ingredients disclosed earlier, the compositions, may and preferably do include other ingredients in order to perform one or more functions of this invention also include a cosmetically acceptable carrier.

Fatty acids having from 10 to 30 carbon atoms may also be suitable as cosmetically acceptable carriers. Illustrative of this category are pelargonic, lauric, myristic, palmitic, stearic, isostearic, oleic, linoleic, linolenic, hydroxystearic and behenic acids and mixtures thereof.

In the cases where the cosmetic composition of the invention is a cream, it is preferred that the cream comprises one or more fatty acids such as stearic or palmitic acid, a part of it neutralised by an alkali. This leads to a mixture of a fatty acid(s) and its soap(s). Such cream compositions are referred to as vanishing creams.

Fatty alcohols having from 10 to 30 carbon atoms are another useful category of cosmetically acceptable carrier. Illustrative of this category are stearyl alcohol, lauryl alcohol, myristyl alcohol, oleyl alcohol and cetyl alcohol and mixtures thereof. Thickeners can be utilized as part of the cosmetically acceptable carrier of compositions according to the present invention. Typical thickeners include crosslinked acrylates (e.g. Carbopol® 982), hydrophobically-modified acrylates (e.g. Carbopol 1382®), polyacrylamides (e.g. Sepigel® 305), acryloylmethylpropane sulfonic acid/salt polymers and copolymers (e.g. Aristoflex® HMB and AVC), cellulosic derivatives and natural gums. Among useful cellulosic derivatives are sodium carboxymethylcellulose, hydroxypropyl methocellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Inorganics may also be utilized as thickeners, particularly clays such as bentonites and hectorites, fumed silicas, talc, calcium carbonate and silicates such as magnesium aluminum silicate (Veegum®). Amounts of the thickener may range from 0.0001 to 10%, usually from 0.001 to 1%, optimally from 0.01 to 0.5% by weight of the composition. Preferred are emollients that can be used, especially for products intended to be applied to the face, to improve sensory properties and are chosen from the group of oils that do not form stiff gels with 12HSA; these include polypropylene glycol-14 butyl ether otherwise known as Tegosoft® PBE, or PPG15 stearyl ether such as Tegosoft® E, other oils such as esters, specifically, isopropyl myristate, isopropyl palmitate, other oils could include castor oils and derivatives thereof.

Skin moisturizers, e.g. hyaluronic acid and/or its precursor N-acetyl glucosamine may be included. N-acetyl glucosamine may be found in shark cartilage or shitake mushrooms and are available commercially from Maypro Industries, Inc (New York). Amounts of the salt may range from 0.2 to 30%, and preferably from 0.5 to 20%, optimally from 1% to 12% by weight of the topical composition, including all ranges subsumed therein.

Ordinarily the alkyl constituent on the quaternized ammonium group will be methyl, ethyl, n-propyl, isopropyl or hydroxyethyl and mixtures thereof. Particularly preferred is a trimethyl ammonium group known through INCI nomenclature as a “trimonium” group. Any anion can be used in the quat salt. The anion may be organic or inorganic with proviso that the material is cosmetically acceptable. Typical inorganic anions are halides, sulfates, phosphates, nitrates and borates. Most preferred are the halides, especially chloride. Organic anionic counter ions include methosulfate, toluoyl sulfate, acetate, citrate, tartrate, lactate, gluconate, and benzenesulfonate.

Still other preferred moisturizing agents which may be used, especially in conjunction with the aforementioned ammonium salts include substituted urea like hydroxymethyl urea, hydroxyethyl urea, hydroxypropyl urea; bis(hydroxymethyl) urea; bis(hydroxyethyl) urea; bis(hydroxypropyl) urea; N,N′-dihydroxymethyl urea; N,N′-di-hydroxyethyl urea; N,N′-di-hydroxypropyl urea; N,N,N′-tri-hydroxyethyl urea; tetra(hydroxymethyl) urea; tetra(hydroxyethyl) urea; tetra(hydroxypropyl urea; N-methyl, N′-hydroxyethyl urea; N-ethyl-N′-hydroxyethyl urea; N-hydroxypropyl-N′-hydroxyethyl urea and N,N′dimethyl-N-hydroxyethyl urea. Where the term hydroxypropyl appears, the meaning is generic for either 3-hydroxy-n-propyl, 2-hydroxy-n-propyl, 3-hydroxy-i-propyl or 2-hydroxy-i-propyl radicals. Most preferred is hydroxyethyl urea. The latter is available as a 50% aqueous liquid from the National Starch and Chemical Division of ICI under the trademark Hydrovance®. Amounts of substituted urea that may be used in the topical composition of this invention range from 0.01 to 20%, and preferably, from 0.5 to 15%, and most preferably, from 2 to 10% based on total weight of the composition and including all ranges subsumed therein.

When ammonium salt and substituted urea are used, in a most especially preferred embodiment at least from 0.01 to 25%, and preferably, from 0.2 to 20%, and most preferably, from 1 to 15% humectant, like glycerine, is used, based on total weight of the topical composition and including all ranges subsumed therein.

### Skin Benefit Ingredients

The inventive compositions preferably include a skin lightening compound, to obtain optimum skin lightening performance at an optimum cost. Illustrative substances are placental extract, lactic acid, niacinamide, arbutin, kojic acid, ferulic acid, hydroquinone, resorcinol and derivatives including 4-substituted resorcinols and combinations thereof. More preferably, such additional skin lightening compound is a tyrosinase inhibitor, to complement the melanogenesis inhibition activity of the substituted monoamines, most preferably a compound selected from the group consisting of kojic acid, hydroquinone and 4-substituted resorcinol. Also, dicarboxylic acids represented by the formula HOOC-(CxHy)-COOH where x=4 to 20 and y=6 to 40 such as azelaic acid, sebacic acid, oxalic acid, succinic acid, fumaric acid, octadecenedioic acid or their salts or a mixture thereof, most preferably fumaric acid or salt thereof, especially di-sodium salt. Combination of 12HSA with fumaric acid or salts thereof are particularly preferred, especially for skin lightening formulations. Amounts of these agents may range from 0.1 to 10%, preferably from 0.5 to 2% by weight of the composition. It is preferred that the skin lightening coactive according to the invention is vitamin B3 or a derivative thereof and is selected from the group consisting of niacinamide, nicotinic acid esters, non-vasodilating esters of nicotinic acid, nicotinyl amino acids, nicotinyl alcohol esters of carboxylic acids, nicotinic acid N-oxide, niacinamide N-oxide and mixtures thereof.

A variety of herbal extracts may optionally be included in compositions of this invention. Illustrative are pomegranate, white birch (Betula alba), green tea, chamomile, licorice and extract combinations thereof. The extracts may either be water soluble or water-insoluble carried in a solvent which respectively is hydrophilic or hydrophobic. Water and ethanol are the preferred extract solvents.

Colorants, opacifiers and abrasives may also be included in compositions of the present invention. Each of these substances may range from 0.05 to 5%, preferably between 0.1 and 3% by weight of the composition.

A rheology modifier may be included and is selected from the group consisting of silica such as fumed silica or hydrophilic silicas and clays such as magnesium aluminum silicate, betonites, hectorite, laponite, and mixtures thereof. A rheology modifier is generally employed in an amount of from 0.01 to 2%, preferably from 0.05 to 1%.

Preservatives could be incorporated into the compositions of this invention to protect against the growth of potentially harmful microorganisms. Suitable traditional preservatives for compositions of this invention are alkyl esters of para-hydroxybenzoic acid. Other preservatives which have come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are iodopropynyl butyl carbamate, phenoxyethanol, caprylyl glycol, C1-6 parabens (especially, methyl paraben and/or propyl paraben), imidazolidinyl urea, sodium dehydroacetate and benzyl alcohol. The preservatives should be selected having regard for the use of the composition and possible incompatibilities between the preservatives and other ingredients in the emulsion.

Preservatives are preferably employed in amounts ranging from 0.01% to 2% by weight of the composition, including all ranges subsumed therein. An especially preferred combination is octocrylene and caprylyl glycol, since caprylyl glycol has been disclosed to enhance UVA and UVB protection. The compositions of the present invention can comprise a wide range of other optional components. The CTFA Cosmetic Ingredient Handbook, Second Edition, 1992, which is incorporated by reference herein in its entirety, describes a wide variety of non-limiting cosmetic and pharmaceutical ingredients commonly used in the skin care industry, which are suitable for use in the compositions of the present invention. Examples include antioxidants, binders, biological additives, buffering agents, colorants, astringents, fragrance, opacifying agents, conditioners, exfoliating agents, pH adjusters, natural extracts, essential oils, skin sensates, skin soothing agents and skin healing agents.

### Method of Using Compositions

The composition according to the invention is intended primarily as a product for topical application to human skin, especially as an agent for protecting from solar radiation, and preventing or reducing the appearance of wrinkled or aged skin, or age spots.

In use, a small quantity of the composition, for example from one to five ml, is applied to exposed area of the skin, from a suitable container or applicator and, if necessary, it is then spread over and/or rubbed into the skin using the hand or fingers or a suitable device.

In accordance with another aspect is disclosed a method of providing abrasion-resistant photoprotection to skin comprising a step of applying a cosmetic composition of the first aspect to the skin.

In accordance with a third aspect is disclosed use of a cosmetic composition according to the first aspect for providing abrasion-resistant photoprotection to skin.

Abrasion resistance is measured by contacting PMMA plates coated with the concerned compositions with sand which is used as an abrasive medium to simulate in use conditions.

The extent of photoprotection is determined by measuring the changes in absorbance and transmittance of UV radiation of the coated PMMA plates after subjecting the plates to abrasion by sand particles.

While the above summarizes the present invention, it will become apparent to those skilled in the art that modifications, variations and alterations may be made without deviating from the scope and spirit of the present invention as described and claimed herein. The invention will be further illustrated in the following non-limiting examples.

### EXAMPLES

A set of cosmetic compositions (face creams) were prepared. Some were within the scope of the present invention while the others were prepared to get comparative data and as reference standards. Details of the compositions are shown in Table 1 and the note below the Table explains further important things about each composition.

TABLE 1

| | Weight % | | | | | |
|---|---|---|---|---|---|---|
| Ingredients | A | B | C | D | E | F |
| Pongamol | 1.2 | 1.2 | 1.2 | 1.2 | — | — |
| Karanjin | 2.4 | 2.4 | 2.4 | 2.4 | — | — |
| Pongamia oil | — | — | — | — | — | 3.6 |
| (commercial grade) | | | | | | |
| Parsol ® MCX | — | — | — | — | 2.4 | — |
| Avobenzone | — | — | — | — | 1.2 | — |
| Stearic acid | 3.0 | 3.0 | 3.0 | 3.0 | 3.0 | 3.0 |
| Mineral Oil | 40.0 | — | — | — | — | — |
| IPM | — | 40.0 | — | — | — | — |
| X-Tend ® 226 | — | — | 40.0 | — | 40.0 | 40.0 |
| (Phenethyl | | | | | | |
| Benzoate) | | | | | | |
| Phenoxy ethanol | — | — | — | 40.0 | — | — |
| KOH (45%) | 1.2 | 1.2 | 1.2 | 1.2 | 1.2 | 1.2 |
| Glyceryl | 3.0 | 3.0 | 3.0 | 3.0 | 3.0 | 3.0 |
| monostearate | | | | | | |
| Glycerin | 1.0 | 1.0 | 1.0 | 1.0 | 1.0 | 1.0 |
| Water + other | 100 | 100 | 100 | 100 | 100 | 100 |
| minors to | | | | | | |

Note:
Composition A did not contain any solvent with Total Hildebrand Parameter (δt) in the range of 20 to 25. It was outside the scope of the invention.
Compositions B contained isopropyl myristate. It was outside the invention.
Compositions C and D were inside the scope of this invention.
Composition E contained two well-known chemical sunscreens but did not contain either pongamol or karanjin. This composition was outside the scope of the invention.
Composition F contained pongamia oil as against pongamol or karanjin. This composition was outside the scope of the invention.

### Abrasion Resistance

This was performed in two stages. In the first stage, transmittance of each formulation was measured before and after abrasion with sand.

In the second stage a fresh lot of PMMA plates coated with the concerned composition was subjected to tests. Absorbance was measured before and after abrasion with sand.

The test methods and the results are described in somewhat details below.

### Measurement of Transmittance Method

To measure abrasion resistance of all the compositions of Table 1, 10 mg of each was applied to one PMMA plate to form a thin film of the composition. Area of the plate was 5 cm² and it contained grooves of 6 μm each. The film was allowed to dry for 30 minutes in the absence of light. Thereafter, initial (0 minute abrasion resistance) transmittance of the plate bearing the film was measured by using SPF-290S Analyser system.

The plate was placed in a sand bath with the film-side of the plates facing the particles of sand. The sand bath was placed on a shaker allowed to run at 120 to 130 RPM for 25 minutes. Thereafter, the plates were removed and tapped to remove any adhering particles of sand. Transmittance was measured again after the (abrasion resistance) treatment as indicated earlier (final transmittance).

The results were expressed in terms of increase in area under the curve (AUC) of transmittance against initial transmittance in the case of each composition. In order to interpret the results properly, the data was converted and expressed in the form of percentage increase in transmittance (AUC) against initial, which was deduced by calculating areas under the curves in each case. It was expected that a technically effective composition would show minimum decrease in the % transmission, and certainly below 20%. The observations are shown in Table 2.

In order to minimise the effects of human or instrumental errors, each experiment was conducted in triplicate and the data shown in Table 2 is the average.

TABLE 2

| Composition no. of | % increase in AUC of Transmittance |
|---|---|
| Table 1 | against the initial transmittance |
| B | 25.0 |
| C | 15.0 |
| D | 5.0 |

Data in Table 2 indicates that transmittance of Composition B (outside the scope of the invention), increased significantly due to abrasion. On the other hand, the transmittance of plates coated with Compositions C and D (both within the scope of the present invention) did not increase noticeably. The data further indicates that abrasion did not appreciably impact the transmittance of the plates coated with compositions C and D.

### Measurement of Absorbance:

To measure the abrasion resistance of the control and inventive compositions, two sets of all the compositions of Table 1 were coated on PMMA plates as done earlier. Ten mg of each was applied to a PMMA plate to form a thin film. Area of the plate was 5 cm² and it contained grooves of 6 μm each. The film was allowed to dry for 30 minutes in the absence of light.

One plate from each set, termed as control plate, was set aside. The plate with the film deposited on such plate was dissolved in chloroform by sonicating such plates for 30 minutes.

All other set of dried plates were subjected to abrasion by sand particles as described earlier. Thereafter, the treated plates with the film deposited on each such plate was dissolved in chloroform using sonication.

As absorbance is always measured against a blank sample, the blank in this case was prepared by dissolving a PMMA plate in chloroform. All the measurements were carried out using Nano-drop Spectrophotometer.

The results were expressed in terms of the decrease in area under the curve (AUC) of absorbance against control (initial minus final) in the case of each composition. In order to interpret the results properly, this data also was converted and expressed in the form of percentage decrease in AUC of absorbance against control absorbance (290 to 400 nm) which was deduced by calculating areas under the curve in each case. It was expected that a technically effective composition would show minimum decrease, and certainly below 20% decrease. These observations are shown in Table 3.

In order to minimise the effects of human or instrumental errors, each experiment was conducted in triplicate and the data shown in Table 3 is the average.

TABLE 3

| Composition no. of Table 1 | % decrease in absorbance |
|---|---|
| A | 28.0 |
| B | 20.0 |
| C | 7.5 |
| D | 9.0 |
| E | — |
| F | — |

Note:
In the case of composition F, the percentage decrease in absorbance could not be measured because the initial absorbance of the film (coated with composition F) was negligible.

Data in Table 3 indicates that absorbance of Compositions A and B (outside the scope of the invention), decreased significantly due to abrasion. On the other hand, the absorbance of Compositions C and D (both within the scope of the present invention) did not decrease noticeably. The absorbance of Composition F which contained 3.6% by weight pongamia oil, was negligible.

The data further indicates that abrasion did not appreciably impact the absorbance of inventive compositions C and D.

1. A cosmetic composition comprising:
(i) 0.1 to 10% by weight pongamol;
(ii) 0.1 to 10% by weight karanjin; and,
(iii) 0.1 to 40% by weight of a solvent whose Total Hildebrand Parameter (δt) value is 18 to 26 MPa^{0.5} at 25° C.,
wherein said composition comprises not more than 2% by weight Pongamia oil.

2. The cosmetic composition of claim 1, wherein said solvent is at least one of phenoxyethanol or phenethyl benzoate.

3. The cosmetic composition of claim 1, wherein w/w ratio of pongamol to karanjin is 1:0.5 to 1:5.

4. The cosmetic composition of as claimed in claim 3, wherein said ratio is 1:1.5 to 1:3.

5. The cosmetic composition of claim 1, wherein said composition is a leave-on composition.

6. The cosmetic composition of claim 5, said composition is a cream or a lotion.

7. The cosmetic composition of claim 6, wherein said cream comprises one or more fatty acids, a part of it neutralised by an alkali.

8. The cosmetic composition as claimed of in claim 1, wherein said composition comprises not more than 2% by weight synthetic organic sunscreens.

9. A method of providing abrasion-resistant photoprotection to skin comprising a step of applying a cosmetic composition of claim 1 to the skin.

10. (canceled)
